(19) United States
(12) Patent Application Publication
(10) Pub. No.: US 2012/0053209 A1
(43) Pub. Date: Mar. 1, 2012

(54) ANTICANCER COMPOUNDS, PREPARATION THEREOF, AND THERAPEUTIC USE THEREOF

(75) Inventors: Jerome Arigon, Paris (FR); Claude Bernhart, Paris (FR); Monsif Bouaboula, Paris (FR); Romain Combet, Paris (FR); Samir Jegham, Paris (FR); Sandrine Hilairet, Paris (FR)

(73) Assignee: SANOFI, Paris (FR)

(21) Appl. No.: 13/258,921

(22) PCT Filed: Mar. 18, 2010
(86) PCT No.: PCT/FR10/50489

§ 371 Date: Nov. 15, 2011

(30) Foreign Application Priority Data
Mar. 24, 2009 (FR) 0901367
Mar. 31, 2009 (FR) 0901589

(51) Int. Cl. A61K 31/444; A61P 35/00; C07D 401/12

(52) U.S. Cl. 514/332; 546/262

(57) ABSTRACT

The invention relates to nicotinamide derivatives that can be used as anticancer drugs.

The present invention relates to novel anticancer derivatives, to the compositions containing them and to the therapeutic use thereof, in particular as anticancer agents. The invention also relates to the process for preparing these compounds and also to some of the intermediate products.

### PRIOR ART

WO 99/31064 describes compounds of formula (A):

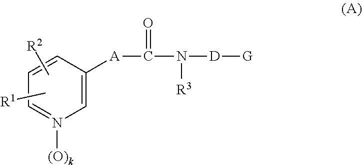

in which A represents in particular an alkylene group in which a methylene unit can be substituted with an O, S, C═O, NH, SO or SO₂ fragment in any position not adjacent to the amide unit —C(═O)—NR₃—. D represents an alkylene, alkenylene or alkinylene group containing at least 3 carbon atoms, in which from 1 to 3 methylene unit(s) may be substituted with an O, S, C═O, NH, SO or SO₂ fragment. G represents in particular the group —(CR₉R₁₀)ₘ—R₈ in which m is 0 or 1. R₉ and R₁₀ can represent a hydrogen atom or an alkyl group, and R₈ represents an aralkyl group or an aromatic or heteroaromatic group which is monocyclic, possibly containing from 1 to 3 heteroatoms (N, O or S) or a bicyclic or tricyclic aromatic group. R₈ may be optionally substituted with: halogen, —ON, alkyl, fluoroalkyl, cycloalkyl, aralkyl, aryl, —OH, hydroxyalkyl, alkoxy (—O-alkyl), aryloxy (—O-aryl), mercapto (—SH), alkylthio (—S-alkyl), arylthio (—S-aryl), carboxyl (—COOH), carboxyalkyl (-alkyl-COOH), carboxyalkenyl (-alkenyl-COOH), alkoxycarbonyl (—COOalkyl), nitro (—NO₂), amino (—NH₂), aminoalkyl (-alkyl-NH₂), monoalkylamino (—NHalkyl), dialkylamino (—N(alkyl)₂). This application neither describes nor suggests the compounds of the invention which comprise a —CH═CH— group on one of the two pyridine nuclei and also the —C(═O)NHR₂ and NR₁R′₁ groups on the other pyridine nucleus.

### DESCRIPTION OF THE INVENTION

### Definitions Used

In the context of the present invention:
the term “halogen atom” is intended to mean: a fluorine, chlorine, bromine or iodine atom;
the term “alkyl group” is intended to mean: a saturated aliphatic hydrocarbon-based group containing from 1 to 6 carbon atoms (advantageously from 1 to 4 carbon atoms) of formula CₙH₂ₙ₊₁—, obtained by removing a hydrogen atom from an alkane. The alkyl group may be linear or branched. By way of examples, mention may be made of methyl, ethyl, propyl, isopropyl, butyl, isobutyl, tert-butyl, pentyl, 2,2-dimethylpropyl and hexyl groups;
the term “alkylene group” is intended to mean: a divalent group of molecular formula —CH₂ₙ—, obtained by removing two hydrogen atoms from an alkane on two different carbon atoms of said alkane;
the term “alkoxy group” is intended to mean: an —O-alkyl group, where the alkyl group is as defined above;
the term “cycloalkyl group” is intended to mean: a cyclic alkyl group containing between 3 and 8 carbon atoms, all the carbon atoms being involved in the cyclic structure. By way of examples, mention may be made of cyclopropyl, cyclobutyl, cyclopentyl or cyclohexyl groups;
the term “heterocycloalkyl group” is intended to mean: a cycloalkyl group comprising at least one heteroatom (O, S, N) involved in the ring and linked to the carbon atoms forming the ring. By way of examples, mention may be made of pyrrolidinyl, piperidinyl, piperazinyl or N—(C₁-C₄)alkylpiperazinyl, azepanyl, thiomorpholinyl, 1-oxo-thiomorpholinyl and 1,1-dioxo-thiomorpholinyl groups.

According to a 1st aspect, a subject of the present invention is a compound of formula (I):

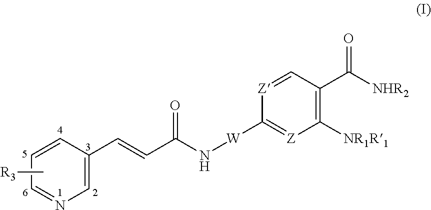

in which:
Z and Z′ represent N or CH;
W represents a —(C₁-C₄)alkylene-CH₂CH₂—, group;
R₁ represents a hydrogen atom, a (C₁-C₆)alkyl group, a (C₃-C₆)cycloalkyl group or a phenyl group;
R′₁ represents a hydrogen atom or a (C₁-C₆)alkyl group;
R₂ represents:
   a (C₃-C₆)cycloalkyl group;
   a (C₁-C₆)alkyl group optionally substituted with:
      one or more hydroxyl or (C₁-C₄)alkoxy groups;
      an —NRₐR_{b} group in which Rₐ and R_{b} represent, independently of one another, a hydrogen atom or a (C₁-C₆)alkyl group or form, together with the nitrogen atom to which they are attached, a (C₄-C₆)heterocycloalkyl group optionally comprising, in the ring, the group —S(O)_{q} with q=0, 1 or 2 or the group —NH— or —N(C₁-C₄)alkyl-, and being optionally substituted with one or more substituent(s), which may be identical to or different from one another when there are several thereof, chosen from an —OH; (C₁-C₄)alkoxy or (C₁-C₄)alkyl group;
R₃ represents at least one substituent of the pyridine nucleus, chosen from a hydrogen or fluorine atom, a (C₁-C₄)alkyl group or —NR_{c}R_{d} in which R_{c} and R_{d} represent a hydrogen atom or a (C₁-C₄)alkyl group.

Z and Z′ represent N or CH. More particularly, Z and Z′ can represent respectively N and CH; CH and CH or N and N

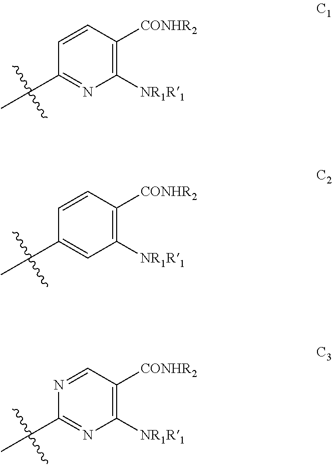

R₁ represents a hydrogen atom, a (C₁-C₆)alkyl group, a (C₃-C₆)cycloalkyl group, for example cyclopropyl, or a phenyl group. R′₁ represents a hydrogen atom or a (C₁-C₆)alkyl group. More particularly, R′₁ represents a hydrogen atom. R₁ and/or R′₁ may be chosen from those described in Table I.

R₂ represents:
a (C₃-C₆)cycloalkyl group, for instance a cyclopropyl or cyclopentyl group;
a (C₁-C₆)alkyl group optionally substituted with:
   one or more —OH or (C₁-C₄)alkoxy, for example methoxy, group(s);
   an —NRₐR_{b} group in which Rₐ and R_{b} represent, independently of one another, a hydrogen atom or a (C₁-C₆)alkyl group or form, together with the nitrogen atom to which they are attached, a (C₄-C₆)heterocycloalkyl group optionally comprising, in the ring, the group —S(O)_{q} with q=0, 1 or 2 or the group —NH— or —N(C₁-C₄)alkyl-. q more particularly represents 1 or 2.

The heterocycloalkyl group formed by Rₐ and R_{b} may, for example, be a pyrrolidinyl

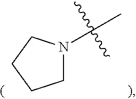

piperidinyl

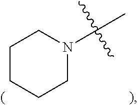

piperazinyl

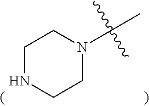

or N—(C₁-C₄)alkylpiperazinyl

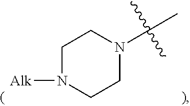

azepanyl

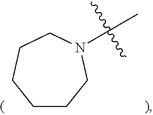

thiomorpholinyl

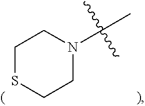

1-oxo-thiomorpholinyl

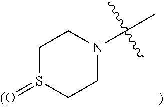

or 1,1-dioxo-thiomorpholinyl

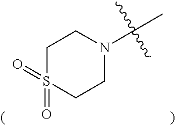

group.

The heterocycloalkyl group formed by Rₐ and R_{b} may be optionally substituted with one or more substituent(s), which may be identical to or different from one another when there are several thereof, chosen from: —OH; (C₁-C₄)alkoxy: for example, methoxy; (C₁-C₄)alkyl: for example, methyl. Thus, the substituted heterocycloalkyl may be a 3-hydroxypiperidinyl

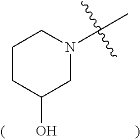

or 4-hydroxypiperidinyl

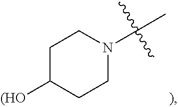

4-methoxypiperidinyl

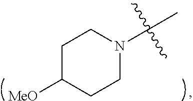

cis-3,5-dimethylpiperidinyl

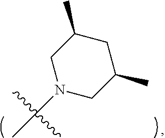

or cis-2,6-dimethylpiperidinyl

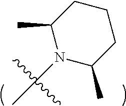

group.

The pyridine nucleus may comprise from 1 to 4 substituents R₃ chosen from a hydrogen or fluorine atom, a (C₁-C₄)alkyl group or —NR_{c}R_{d} in which R_{c} and R_{d} represent a hydrogen atom or a (C₁-C₄)alkyl group. Preferably. R₃ is in position 5 and/or 6 on the pyridine nucleus. Preferably, the number of substituents R₃ is equal to 1 and/or R₃ is in position 5 or 6 on the pyridine nucleus as is represented below:

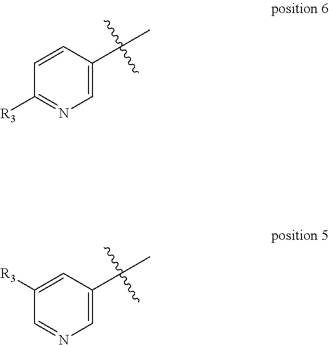

R₃ is even more preferably in position 6. Preferably, R₃ represents a hydrogen atom or —NH₂.

W represents a —(C₁-C₄)alkylene-CH₂CH₂— group, in particular the —(CH₂)ₘ— group, m being an integer between 1 and 6.

The subgroup of formula (I′) is differentiated:

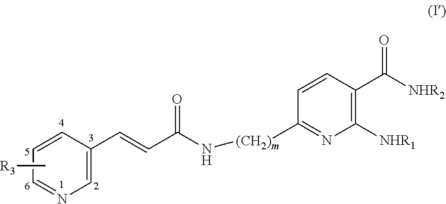

in which R₁ represents a (C₁-C₆)alkyl group, R₂ represents a (C₁-C₆)alkyl group optionally substituted with an —NRₐR_{b} group in which Rₐ and R_{b} represent, independently of one another, a hydrogen atom or a (C₁-C₆)alkyl group or form, together with the nitrogen atom to which they are attached, a (C₄-C₆)heterocycloalkyl group optionally comprising, in the ring, the group —S(O)_{q} with q=0, 1 or 2 or the group —NH— or —N(C₁-C₄)alkyl, and R₃ represents a hydrogen atom or an —NR_{c}R_{d} group as defined above, positioned in position 5 or 6 on the pyridine nucleus, and m is an integer between 1 and 6.

The double bond on the pyridine nucleus may be in E or Z form. Preferably, they are in E form.

The compounds of the invention, including the exemplified compounds, can exist in the form of bases or of addition salts with acids. Such addition salts are also part of the invention. These salts are advantageously prepared with pharmaceutically acceptable acids, but the salts of other acids that are useful, for example, for purifying or isolating the compounds are also part of the invention. The compounds according to the invention may also exist in the form of hydrates or of solvates, i.e. in the form of associations or combinations with one or more molecules of water or with a solvent. Such hydrates and solvates are also part of the invention.

The compounds may comprise one or more asymmetrical carbon atoms. They may therefore exist in the form of enantiomers or of diastereoisomers. These enantiomers and diastereoisomers, and also mixtures thereof, are part of the invention.

According to a 2nd aspect, a subject of the invention is the process for preparing the compounds of the invention and also some of the reaction intermediates.

### Preparation of the Compounds of Formula (I)

These compounds can be prepared according to Scheme 1.

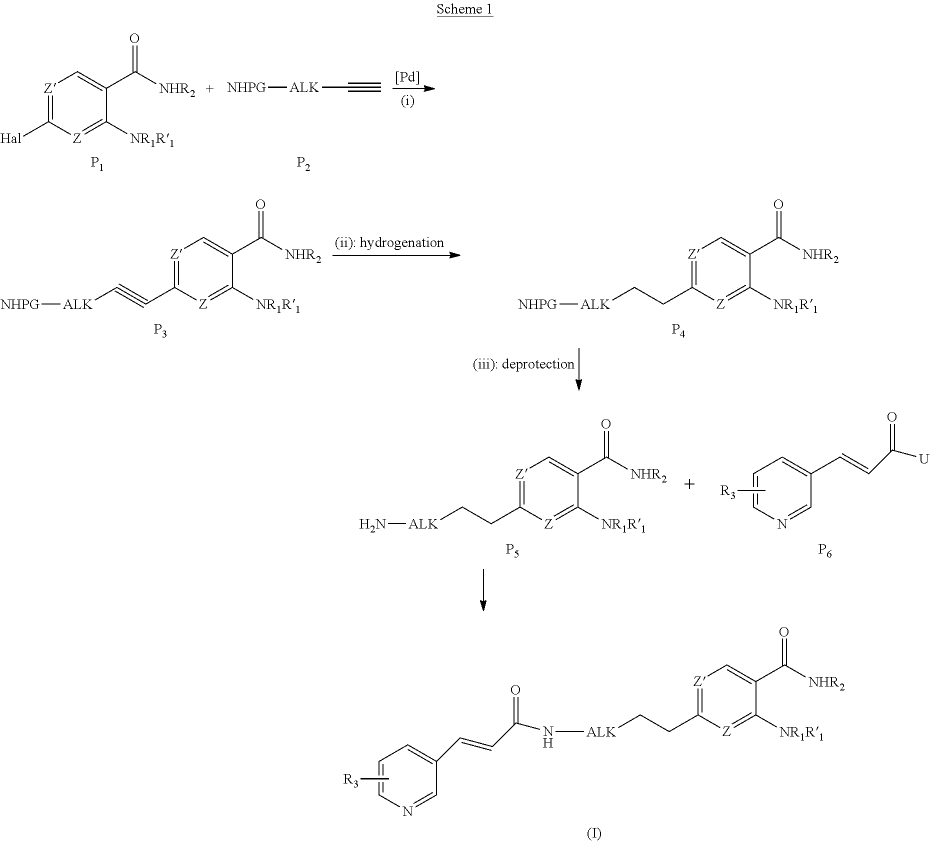

In stage (i). Sonogashira coupling is carried out between P₁ and P₂ so as to obtain P₃. Hal represents a halogen (chlorine, bromine, iodine) atom. ALK represents a (C₁-C₄)alkylene group, in particular the group —(CH₂)ₘ—. PG represents an amine-function-protecting group, for example BOC, and U represents OH or a halogen atom such as chlorine. The coupling is carried out in the presence of a palladium (in the oxidation state (0) or (II)) complex in a solvent in a basic medium. The palladium complex may, for example, be Pd(PPh₃)₄, PdCl₂(PPh₃)₂, Pd(OAc)₂, PdCl₂(dppf) or bis(di-tert-butyl(4-dimethylaminophenyl)phosphine)-dichloropalladium(II).

A copper (I) salt, such as cuprous chloride, is generally required as a cocatalyst of the palladium complex. However, it has recently been discovered that certain catalytic systems do not require a copper salt, for instance the system Pd₂(dba)₃, P(t-Bu)₃ or Et₃N in THF (Eur. J. Org. Chem. 2000, 3679).

It is preferable to carry out the process in a deoxygenated medium so as to preserve the catalytic system when the latter is sensitive to oxygen.

The coupling is carried out in the presence of a basic medium, which may be, for example, K₂CO₃, NaHCO₃, Et₃N, K₃PO₄, Ba(OH)₂, NaOH, KF, CsF, Cs₂CO₃, etc. The coupling may be carried out in a mixture of a polar solvent, for example DMF. The temperature is between 50 and 120° C. The reaction time may in certain cases exceed be long (see conditions of Ex. 1.).

Further details on the Sonogashira coupling (Scheme 1 of Chem. Rev.) and on the operating conditions and the palladium complexes, copper salts and bases that can be used, will be found in: Chem. Rev. 2007, 107(3), 874; Tetrahedron Lett. 2007, 48, 7129-7133; K. Sonogashira in “Metal-Catalyzed Cross-coupling Reactions”, 1998, eds. F. Diederich, P. J. Stang, Wiley-VCH, Weinheim, ISBN 3-527-29421-X.

In stage (ii), the —C≡C bond is hydrogenated. It is possible to use hydrogen in the presence of a metal catalyst, for example palladium deposited on a solid support (for example, Pd/C). The hydrogenation may be carried out, for example, at ambient temperature, with hydrogen under a pressure of the order of 1 atm in the presence of palladium-on-charcoal, for a period of the order of 20-30 min. See, for example, the conditions of Ex. 1.5. Other techniques for hydrogenating —C≡C— bonds so as to give —CH₂CH₂— bonds exist and are known to those skilled in the art.

In stage (iii), P₄ is deprotected, for example by means of a treatment in an acidic medium when PG represents BOC.

In stage (iv), P₅ and P₆, which is either an acid (U=OH) or an acyl halide (U=Br, Cl or F), are reacted (amidation reaction). When U represents an acid, the amidation can advantageously be carried out in the presence of an acid activator (also known as “coupling agent”), for instance benzotriazol-1-yloxytris(dimethylamino)phosphonium hexafluorophosphate (or BOP, CAS No. 56602-33-6, see also Castro, B., Dormoy, J. R. Tetrahedron Letter 1975, 16, 1219) or (O-benzotriazol-1-yl)-N,N,N′,N′-tetramethyluronium tetrafluoroborate (TBTU).

According to a variant of Scheme 1, it is also possible to invert the two hydrogenation and deprotection stages.

### Preparation of P₁

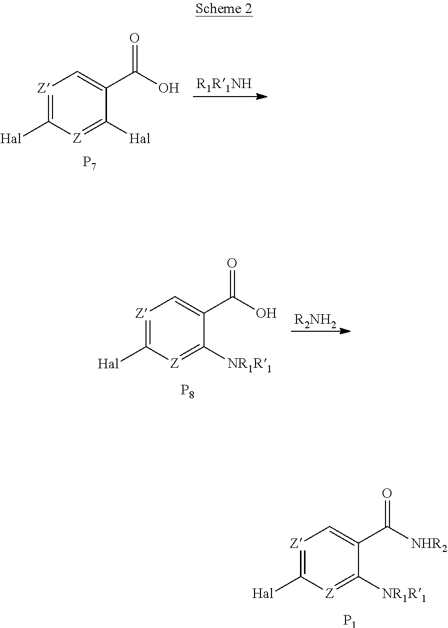

P₈ is obtained starting from the acid P₇ by monosubstitution with an amine of formula R₁R′₁NH. In the case of an aliphatic or cycloaliphatic amine, the reaction can be carried out at ambient temperature and in a protic solvent such as an alcohol or water, or in an aprotic solvent such as THF (see also Ex. 1.1.). In the case of an aniline, a strong base such as, for example, LiHMDS (((CH₃)₃Si)₂NLi) is added and the reaction is carried out under hot conditions. The monosubstitution is described on pages 14-15 of FR 2917412 in the case where Z=N and Z′=CH, but can apply to other Z/Z′.
Z=N, Z′=CH: P₇ is a 2,6-dihalonicotinic acid, for example 2,6-dichloronicotinic acid, which is commercially available;
Z=N, Z′=N: P₇ is a 2,4-dihalopyrimidine carboxylic acid, for example 2,4-dichloropyrimidine carboxylic acid, which is commercially available (CAS No. 37131-89-8);
Z=CH, Z′=CH: P₇ is a 2,4-dihalobenzoic acid, for example 2,4-dichlorobenzo c acid, which is commercially available (CAS No. 50-84-0).

In the case where Z and Z′ both represent N, and Hal represents a chlorine atom. P₈ may also be obtained starting from the commercially available compound ethyl 2,4-dichloro-pyrimidine-5-carboxylate:

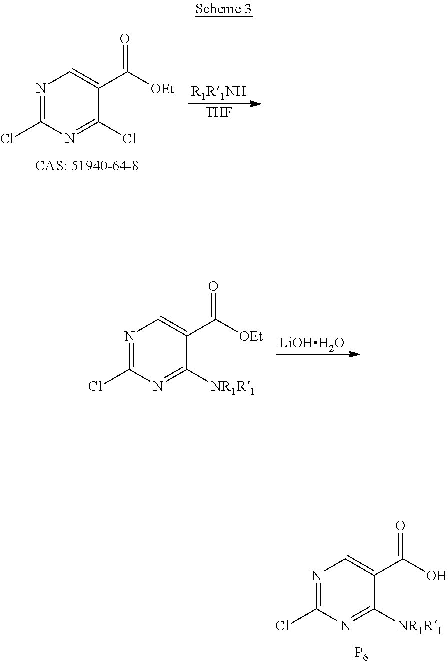

Scheme 3 using an ester function subsequently converted to an acid function also applies to the case where Z=N and Z′═CH: see the conditions in Chem. Pharm. Bull, 2000, 48(12), 1847-1853 (reactions in Tables 1 and 2). P6

P₁ is obtained starting from the acid in P₈ by amidation using the amine R₂NH₂ or a salt of this amine, for example the hydrochloride. The amidation can be carried out advantageously in the presence of an acid activator (also called coupling agent), for instance benzotriazol-1-yloxytris(dimethylamino)phosphonium hexafluorophosphate (or BOP, CAS No. 56602-33-6, see also Castro, B., Dormoy, J. R. Tetrahedron Letter 1975, 16, 1219). The reaction is preferably carried out in the presence of a base (such as triethylamine) at ambient temperature, in a solvent such as tetrahydrofuran (THF) or dimethylformamide (DMF).

### Preparation of P₂

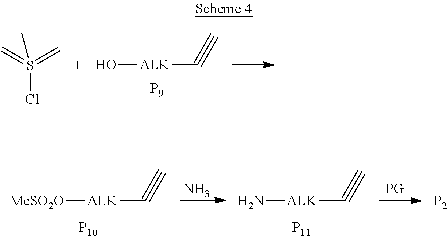

P₂ is obtained starting from P₉ according to Scheme 4 by conversion of the alcohol function to give an amine function via the intermediate P₁₀ carrying the mesyl leaving group, and then protection of P₁₁ with PG. Sodium azide can also be used in place of NH₃ so as to give an azide function, which is subsequently converted to give an amine function (see scheme II of Tetrahedron 1987, 43(21), 5145-58 and scheme 1 of Tetrahedron 2008, 64, 3578-3588).

### R₂NH₂ Compounds

The R₂NH₂ amines are commercially available products or products already described in published documents:
1-(2-aminoethyl)piperidine: CAS No. 27578-60-5, described in Justus Liebigs Annalen der Chemie 1950, 566, 210-44, sold by Acros;
1-(2-aminoethyl)-4-piperidinol: CAS No. 129999-60-6, described in J. Med. Chem. 2005, 48(21), 6690-6695;
1-(2-aminoethyl)-3-piperidinol: CAS No. 847499-95-0, described in J. Med. Chem. 2005, 48(21), 6690-6695;
2-(4-methoxy-1-piperidinyl)ethylamine: CAS No. 911300-69-1, described in J. Med. Chem. 2007, 50(20), 4818-4831;
pyrrolidineethanamine: CAS No. 7154-73-6, described in Anales de Quimica 1974, 70(9-10), 733-737, sold by International Laboratory Ltd, 1067 Sneath Ln, San Bruno, Calif. 94066, USA;
azepan-1-ylethylannine: CAS No. 51388-00-2, described in Anales de Quimica 1974, 70(9-10), 733-737;
2-(1,1-dioxothiomorpholin-4-yl)ethylamine: CAS No. 89937-52-0, sold by Intern. Lab. Ltd;
N-(2-aminoethyl)thiamorpholine-1-oxide: CAS No. 1017791-77-3, sold by Sinova Inc. 3 Bethesda Metro Center, Suite 700, Bethesda, Md., 20814, USA.

A method for obtaining the compounds for which R₂ represents a (C₁-C₆)alkyl group substituted with the —NRₐR_{b} group in which Rₐ and R_{b} form, together with the nitrogen atom to which they are attached, the (C₄-C₆)heterocycloalkyl group optionally comprising, in the ring, the group —S(O)_{q} with q=0, 1 or 2 or the group —NH— or —N(C₁-C₄)alkyl, is described in Scheme 5 and is based on scheme 3 of Bioorg. Med. Chem. 2007, 15, 365-373 or on scheme 2 of Bioorg. Med. Chem. Lett. 2008, 18, 1378-1381:

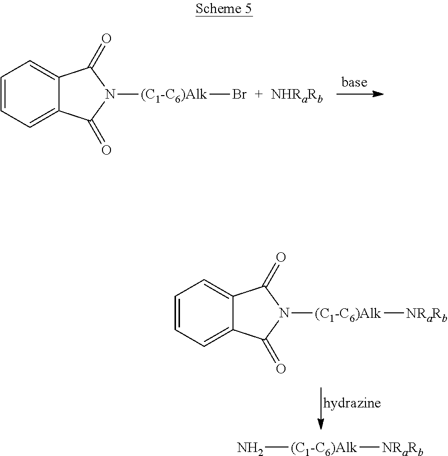

Another method described in Scheme 6 is based on FIG. 2 of Bioorg. Med. Chem. Lett. 2006, 16, 1938-1940:

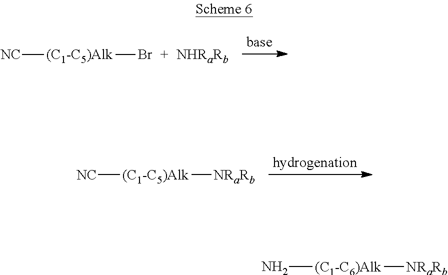

### P₆ Compounds

P₆ with U=OH may be either commercially available or prepared according to the methods known to those skilled in the art. For example, trans-3-(3-pyridyl)acrylic acid is sold by Sigma-Aldrich. (6-Aminopyridin-3-yl)acrylic acid (CAS No. 234098-57-8; compound E: CAS No. 167837-43-6) is described in J. Med. Chem. 2002, 45(15), 3246-3256 (see Scheme 4). P₆ can be prepared starting from a bromoaniline and acrylic acid according to the teaching of J. Med. Chem. 2002, 45(15), 3246-3256. It is also possible to use coupling using a bromo-aniline and an alkyl acrylate, and then to saponify the ester function so as to give an acid function (in this respect, see the method for preparing (6-aminopyridin-3-yl)acrylic acid described in paragraph [483] of US 2008269220 or [354] of EP1726580).

P₆ can also be prepared according to J. Org. Chem. 1998, 63, 8785-8789 starting from the corresponding beta-formylpyridine or else according to J. Med. Chem. 1989, 32(3), 583-93 starting from 2-chloro-5-nitropyridine.

P₆ with U=Hal (acyl halide) is obtained by means of a reaction known to those skilled in the art, starting from the acid P₆ with U=OH and an acylating agent, for instance SOCl₂ or (COCl)₂.

### P₉ Compounds

P₉ may be either commercially available (for example, 3-butyn-1-ol CAS No. 927-74-2 or 2-propyn-1-ol CAS No. 107-19-7) or prepared according to the methods known to those skilled in the art.

### Protection of the Primary or Secondary Amine Function

It may be necessary to use, in at least one of the stages, a protective group (PG) in order to protect one or more chemical function(s), in particular a primary or secondary amine function. For example, when Rₐ and R_{b} both represent a hydrogen atom, the amidation in Scheme 2 is carried out using, for R₂NH₂, the compound ₂HN—(C₁-C₆)alkyl-NH-PG, where PG advantageously represents BOC (tert-butoxycarbonyl). Likewise, when the heterocycloalkyl group formed by Rₐ and R_{b} represents the piperazinyl group

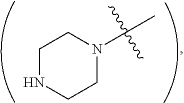

the —NH— function thereof can be advantageously protected using the following compound

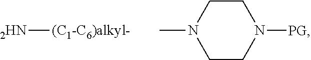

where PG advantageously represents BOC. Likewise, when R₃ represents the —NH₂ or —NHR_{c} group, the amine function can be advantageously protected with one or two PG group(s), preferably BOC. Use may, for example, be made of the following compound

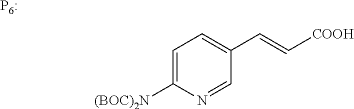

The chemical function(s) is (are) subsequently obtained by means of a (final or intermediate) deprotection stage, the conditions of which depend on the nature of the function(s) protected and on the protective group used. Reference may be made to “Protective groups in Organic Synthesis” by T. Greene, Wiley, 4^{th} ed., ISBN=978-0-471-69754-1, in particular to chapter 7 as regards amine-function-protecting groups. In the case of the protection of —NH₂ or —NH— functions with BOC, the deprotection stage is carried out in an acidic medium using, for example. HCl or trifluoroacetic acid (TFA). The associated salt (hydrochloride or trifluoroacetate) is thus, where appropriate, obtained.

### Obtaining of Salts

The salts are obtained during the deprotection stage described above or else by bringing the acid into contact with the compound in its base form.

In the above schemes, the starting compounds and the reactants, when the method for preparing them is not described, are commercially available or described in the literature, or else can be prepared according to methods which are described therein and which are known to those skilled in the art. Those skilled in the art may also use as a basis the operating conditions given in the examples which are described hereinafter.

According to a 3rd aspect, the invention concerns a pharmaceutical composition comprising a compound as defined above, in combination with a pharmaceutically acceptable excipient. The excipient is chosen from the usual excipients known to those skilled in the art, according to the pharmaceutical form and the method of administration desired. The method of administration may, for example, be oral or intravenous administration.

According to a 4th aspect, a subject of the invention is a medicament which comprises a compound as defined above, and also the use of a compound as defined above, for the manufacture of a medicament. It may be useful for treating a pathological condition, in particular cancer. The medicament (and also a compound according to the invention) may be administered in combination with one (or more) anticancer agent(s). This treatment may be administered simultaneously, separately or else sequentially. The treatment will be adapted by the practitioner according to the disease and the tumour to be treated.

According to a 5th aspect, the invention also concerns a method for treating the pathologies indicated above, which comprises the administration, to a patient, of an effective dose of a compound according to the invention or a pharmaceutically acceptable salt or hydrate or solvate thereof.

### EXAMPLES

The following examples illustrate the preparation of some compounds in accordance with the invention. The numbers of the compounds exemplified refer back to those given in the table hereinafter, which illustrates the chemical structures and the physical properties of some compounds according to the invention.

The compounds were analyzed by HPLC-UV-MS coupling (liquid chromatography, ultraviolet (UV) detection and mass detection). The apparatus used is composed of an Agilent chromatography system equipped with an Agilent diode array detector and with a Waters ZQ single quadripole mass spectrometer or a Waters Quattro-Micro triple quadripole mass spectrometer.

### Mass Spectrometry Conditions

The liquid chromatography/mass spectrometry (LC/MS) spectra were recorded in positive electrospray (ESI) mode, in order to observe the ions resulting from the protonation of compounds analyzed (MH⁺) or from the formation of adducts with other cations, such as Na⁺. K⁺, etc. The ionization parameters are as follows: cone voltage: 20 V; capillary voltage: 3 kV; source temperature: 120° C.; desolvation temperature: 450° C.; desolvation gas: N₂ at 450 l/h.

The HPLC conditions are chosen from one of the following methods:

| Conditions | TFA15 | TFA3 |
|---|---|---|
| Column | Symmetry C18 | Acquity BEH C18 |
| | (50 × 2.1 mm; 3.5 μm) | (50 × 2.1 mm; 1.7 μm) |
| Eluent A | H₂O + TFA 0.005% at | H₂O + TFA 0.05% at |
| | approximately pH 3.1 | approximately pH 3.1/CH₃CN |
| | | (97/3) |
| Eluent B | CH₃CN + TFA 0.005% | CH₃CN + TFA 0.035% |
| Gradient | 100:0 (0 min) [symbol] 10:90 | 100:0 (0 min) [symbol] 5:95 (2.3 min) [symbol] |
| A:B | (10 min) [symbol] 100:0 (15 min) | 5:95 (2.9 min) [symbol] 100:0 (3 min) [symbol] |
| | | 100:0 (3.5 min) |
| T_{column} | 30° C. | 40° C. |
| Flow rate | 0.4 ml/min | 1 ml/min |
| Detection | λ = 220 nm | λ = 220 nm |

TFA: trifluoroacetic acid

### NMR Conditions

The ¹H NMR spectra are recorded on a Bruker Avance 250/Bruker Avance 400 or Bruker Avance II 500 spectrometer. The central peak of DMSO-d6 (2.50 ppm) is used as an internal reference. The following abbreviations are used: s: singlet; d: doublet; dd: double of doublets; t: triplet; q: quadruplet; m; unresolved peak/multiplet; br. s: broad signal.

### Example 1

### 1.1. 6-chloro-2-ethylaminonicotinic acid

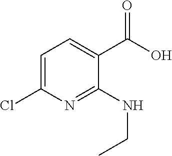

In a round-bottomed flask, 26.1 g (0.136 mol) of 2,6-dichloronicotinic acid are mixed with 180 ml of an aqueous solution of ethylamine at 70%. The mixture is stirred at ambient temperature (AT) for 5 d. The mixture is evaporated under reduced pressure (RP). The residue is taken up with 100 ml of water. The resulting product is cooled using an ice bath and acidified to pH=3 with a 5N HCl solution. The precipitate is filtered off, washed with cold water and vacuum-dried over P₂O₅ at 60° C. 24.93 g (91.4%) of a white solid are obtained. Mp=157-159° C.

### 1.2. 6-Chloro-2-ethylamino-N-methylnicotinamide

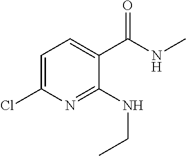

In a round-bottomed flask, 5.0 g (24.92 mmol) of 6-chloro-2-ethylaminonicotinic acid are dissolved in 300 ml of THF. 10.41 ml (74.77 mmol) of triethylamine are added, followed by 14.95 ml (29.91 mmol) of a 2N solution of methylamine in THF and subsequently 13.22 g (29.91 mmol) of BOP. The mixture is stirred at AT for 15 h. The solvent is evaporated off and the residue is taken up with ethyl acetate. The organic phase is washed with water and then a saturated solution of NaCl. The resulting product is dried over Na₂SO₄, filtered and evaporated. The residue is purified by flash chromatography (gradient DCM-MeOH 1 to 10%). 4.1 g are obtained (yield: 77%) (LCMS-LS: tr=1.19 min).

### 1.3. [4-(6-Ethylamino-5-methylcarbamoylpyridin-2-yl)but-3-ynyl]carbamic acid tert-butyl ester

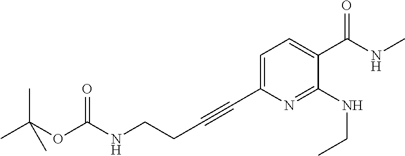

2.9 g (3 mmol) of 6-chloro-2-ethylamino-N-methylnicotinamide are dissolved in 20 ml of DMF. 2.29 g (13.57 mmol) of tert-butyl but-3-yn-1-ylcarbamate and 6.61 ml (47.50 mmol) of triethylamine are added. The mixture is degassed with argon for 30 min and then 0.47 g (0.68 mmol) of dichlorobis(triphenylphosphine)palladium(II) and 0.13 g (0.068 mmol) of CuI are added. The mixture is stirred while heating at 90° C. for 12 h. The mixture is evaporated and the residue is taken up with CH₂Cl₂ (DCM); the resulting product is washed with water and dried over sodium sulphate; the resulting product is filtered and evaporated. The residue is purified by flash chromatography with 100 DCM/90-10 DCM-MeOH. 2.5 g are obtained (yield=45%); LCMS-LS: tr=2.15 min.

### 1.4. 6-(4-Aminobut-1-ynyl)-2-ethylamino-N-methylnicotinamide

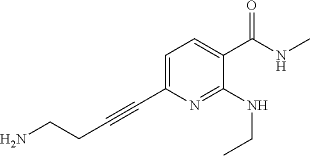

2.5 g (7.22 mmol) of [4-(6-ethylamino-5-methylcarbamoylpyridin-2-yl)but-3-ynyl]carbamic acid tert-butyl ester are dissolved in 30 ml of DCM. The mixture is cooled using an ice bath and 11.12 ml of TFA are added. The mixture is stirred at AT for 15 h. The solvent is evaporated off. The residue is purified by flash chromatography with 100 DCM/80-20 DCM-MeOH. 0.8 g is obtained (yield=45%); LCMS-LS: tr=1.62 min.

### 1.5. 6-(4-Aminobutyl)-2-ethylamino-N-methylnicotinamide

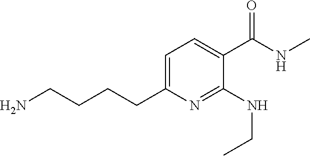

0.8 g (3.25 mmol) of 6-(4-aminobut-1-ynyl)-2-ethylamino-N-methylnicotinamide is dissolved in 50 ml of ethanol and the mixture is hydrogenated at AT and under normal pressure in the presence of 0.07 g (0.06 mmol) of Pd/C at 10%. The resulting product is filtered through Whatman paper and the filtrate is evaporated off. 0.76 g is obtained (yield=93.8%) (LCMS-LS: tr=1.52 min)

### 1.6. Ethyl (2E)-3-{6-[bis(tert-butoxycarbonyl)amino]pyridin-3-yl}acrylate

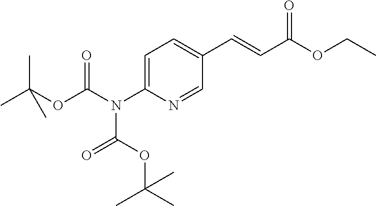

1 g (5.2 mmol) of (E)-3-(6-aminopyridin-3-yl)acrylic acid ethyl ester is dissolved in 50 ml of THF. 4.78 ml (34.34 mmol) of triethylamine and 1.65 g (13.01 mmol) of DMAP are added. The mixture is cooled to 0° C. and then 2.83 g (13.01 mmol) of Boc₂O, dissolved beforehand in 5 ml of THF, are added dropwise. The mixture is brought back to AT, stirred for 1 h and then heated at 75° C. for 6 h. The mixture is evaporated and the residue is taken up with DCM; the resulting product is washed with water and dried over sodium sulphate; filtration and evaporation are carried out. The residue is purified by flash chromatography with 100 DCM/95-5 DCM-MeOH. 1.6 g are obtained (yield=80%).

### 1.7. (2E)-3-{6-[bis(tert-butoxycarbonyl)amino]pyridin-3-yl}acrylic acid

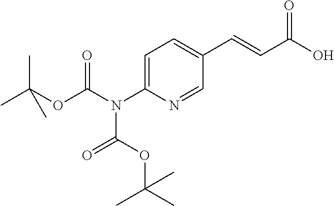

1.2 g (3.06 mmol) of ethyl (2E)-3-{6-[bis(tert-butoxycarbonyl)amino]pyridin-3-yl}acrylate are dissolved in 50 ml of dioxane, and 0.22 g (9.17 mmol) of LiOH, dissolved beforehand in 1 ml of water, is added. The mixture is heated at 70° C. for 3 h. The mixture is concentrated and taken up in a minimum amount of water, and then a 1N solution of HCl is added dropwise until the compound precipitates. The precipitate is filtered off, washed with cold water and vacuum-dried over P₂O₅ at 60° C. 0.9 g of a white solid is obtained (81%).

### 1.8. 6-{4-[(2E)-3-{6-[bis(tert-butoxycarbonyl)amino]pyridin-3-yl}acryloylamino]butyl}-2-ethylamino-N-methylnicotinamide

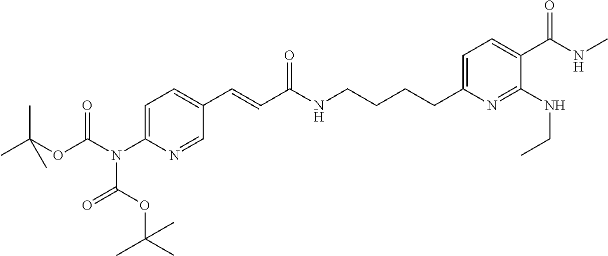

In a round-bottomed flask, 0.59 g (1.39 mmol) of 6-(4-aminobutyl)-2-ethylamino-N-methyl-nicotinamide is dissolved in 20 ml of THF. 0.48 ml (3.48 mmol) of triethylamine is added, followed by 0.29 g (1.16 mmol) of (2E)-3-{6-[bis(tert-butoxycarbonyl)amino]pyridin-3-yl}acrylic acid and then 0.47 g (139 mmol) of BOP. The mixture is stirred at AT for 15 h. The solvent is evaporated off and the residue is taken up with DCM. The organic phase is washed with water and then a saturated solution of NaCl. The resulting product is dried over Na₂SO₄, filtered and evaporated. The residue is purified by flash chromatography (gradient of DCM-MeOH 1% to 15%). 0.38 g is obtained (yield; 55%).

### 1.9. 6-{4-[(E)-3-(6-Aminopyridin-3-yl)acryloylamino]butyl}-2-ethylamino-N-methyl-nicotinamide

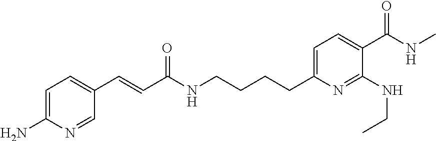

0.3 g (0.5 mmol) of 6-{4-[(2E)-3-{6-[bis(tert-butoxycarbonyl)amino]pyridin-3-yl}acryloylamino]-butyl}-2-ethylamino-N-methylnicotinamide is dissolved in DCM; the mixture is cooled using an ice bath and 30 eq of trifluoroacetic acid (TFA) are added. The resulting mixture is stirred at AT for 12 h. The mixture is evaporated and the residue is taken up with a 10% Na₂CO₃ solution. The precipitate is filtered off and washed with water. The resulting product is vacuum-dried over P₂O₅ at 60° C. 0.17 g is obtained (yield=8996). LCMS (TFA3) m/z=397 tr=0.57 min.

¹H NMR (250 MHz, DMSO-d6) δ ppm 1.13 (t, 3H), 1.37-1.54 (m, 2H), 1.59-1.78 (m, 2H), 2.56 (t, 2H), 2.72 (d, 3H), 3.17 (q, 2H), 3.33-3.48 (m, 2H), 6.26-6.60 (m, 5H), 7.26 (d, 1H), 7.58 (d, 1H), 7.77 (d, 1H), 7.88 (t, 1H), 8.06 (s, 1H), 8.18-8.43 (m, 2H).

The following three other compounds are prepared in a manner identical to Example 1:

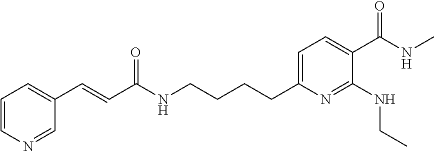

### Example 2

[LCMS (TFA15) m/z=382 tr=4.83 min; ¹H NMR (250 MHz, DMSO-d6) δ ppm 1.07 (t, 3H), 1.31-1.51 (m, 2H), 1.55-1.73 (m, 2H), 2.52 (t, 2H), 2.67 (d, 3H), 3.16 (q, 2H), 3.28-3.41 (m, 2H), 6.36 (d, 1H), 6.67 (d, 1H), 7.33-7.52 (m, 2H), 7.73 (d, 1H), 7.92 (d, 1H), 8.11 (t, 1H), 8.20-8.37 (m, 2H), 8.51 (d, 1H), 8.71 (d, 1H)].

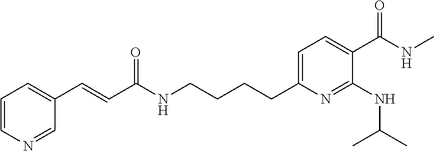

### Example 3

[LCMS (TFA15) m/z=396 tr=5.05 min; ¹H NMR (400 MHz, DMSO-d6) δ ppm 1.15 (d, 6H), 1.42-1.54 (m, 2H), 1.61-1.75 (m, 2H), 2.58 (t, 2H), 2.72 (d, 3H), 3.21 (q, 2H), 4.13-4.24 (m, 1H), 6.39 (d, 1H), 6.72 (d, 1H), 7.39-7.49 (m, 2H), 7.77 (d, 1H), 7.91-7.99 (m, 1H), 8.15 (t, 1H), 8.26-8.38 (m, 2H), 8.55 (dd, 1H), 8.75 (d, 1H)].

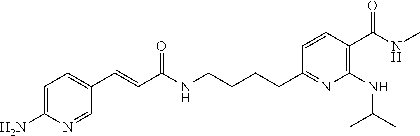

### Example 4

[LCMS (TFA3) m/z=411 tr=0.63 min; ¹H NMR (250 MHz, DMSO-d6) δ ppm 1.15 (d, 6H), 1.38-1.53 (m, 2H), 1.59-1.77 (m, 2H), 2.56 (t, 2H), 2.71 (d, 3H), 3.17 (q, 2H), 4.09-4.27 (m, 1H), 6.32 (d, 1H), 6.36 (s, 2H), 6.39 (d, 1H), 6.47 (d, 1H), 7.25 (d, 1H), 7.58 (dd, 1H), 7.77 (d, 1H), 7.88 (t, 1H), 8.05 (d, 1H), 8.24-8.38 (m, 2H)].

These four compounds were subjected to pharmacological tests for determining the anticancer activity. They were tested in vitro on the following tumour lines: HCT116 (ATCC-CCL247) and PC3 (ATCC-CRL1435). The proliferation and the cell viability were determined in a test using 3-(4,5-dimethylthiazol-2-yl)-5-(3-carboxymethoxyphenyl)-2-(4-sulphophenyl)-2H-tetrazolium (MTS) according to Fujishita T. et al., Oncology 2003, 64(4), 399-406. In this test, the mitochondrial capacity of the living cells for converting MTS into a coloured compound after incubation of the tested compound for 72 hours is measured. IC₅₀ denotes the concentration of compound which results in a 50% loss of proliferation and of cell viability.

For these four compounds, an IC₅₀<10 nM was found on the HCT116 and PC3 lines.

1. A compound of formula (I): 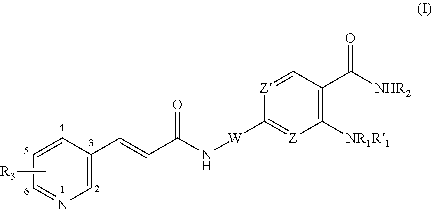
in which:
Z and Z′ represent N or CH;
W represents a —(C₁-C₄)alkylene-CH₂CH₂— group;
R₁ represents a hydrogen atom, a (C₁-C₆)alkyl group, a (C₃-C₆)cycloalkyl group or a phenyl group;
R′₁ represents a hydrogen atom or a (C₁-C₆)alkyl group;
R₂ represents:
a (C₃-C₆)cycloalkyl group;
a (C₁-C₆)alkyl group optionally substituted with:
one or more hydroxyl or (C₁-C₄)alkoxy groups;
an —NRₐR_{b} group in which Rₐ and R_{b} represent, independently of one another, a hydrogen atom or a (C₁-C₆)alkyl group or form, together with the nitrogen atom to which they are attached, a (C₄-C₆)heterocycloalkyl group optionally comprising, in the ring, the group —S(O)_{q} with q=0, 1 or 2 or the group —NH— or —N(C₁-C₄)alkyl-, and
being optionally substituted with one or more substituent(s), which may be identical to or different from one another when there are several thereof, chosen from an —OH; (C₁-C₄)alkoxy or (C₁-C₄)alkyl group;
R₃ represents at least one substituent of the pyridine nucleus, chosen from a hydrogen or fluorine atom, a (C₁-C₄)alkyl group or —NR_{c}R_{d} in which R_{c} and R_{d} represent a hydrogen atom or a (C₁-C₄)alkyl group.

2. The compound according to claim 1, wherein R′₁ represents a hydrogen atom.

3. The compound according to claim 1, in which the (C₄-C₆)heterocycloalkyl group formed by the —NRₐR_{b} group is chosen from pyrrolidinyl 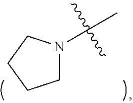
piperidinyl 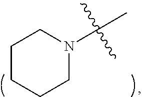
piperazinyl 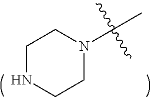
or N—(C₁-C₄)alkylpiperazinyl 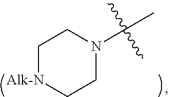
azepanyl 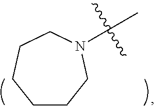
1-oxo-thiomorpholinyl 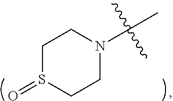
1,1-dioxo-thiomorpholinyl 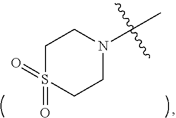
3-hydroxypiperidinyl 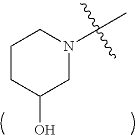
or 4-hydroxypiperidinyl 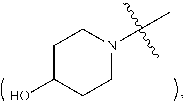
4-methoxypiperidinyl 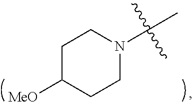
cis-3,5-dimethylpiperidinyl 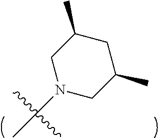
or cis-2,6-dimethylpiperidinyl

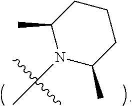

4. The compound according to claim 1, in which R₃ is in position 5 and/or 6 on the pyridine nucleus.

5. The compound according to claim 1, in which the number of substituents R₃ is equal to 1 and/or R₃ is in position 5 or 6 on the pyridine nucleus.

6. The compound according to claim 1, in which R₃ is H or —NH₂.

7. The compound according to claim 1, in which W represents —(CH₂)ₘ—, m being an integer between 1 and 6.

8. The compound according to claim 1, of formula (I′): 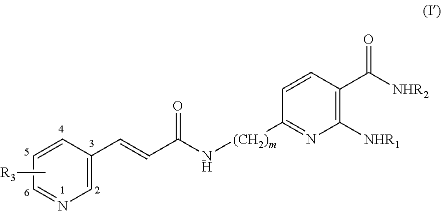
in which R₁ represents a (C₁-C₆)alkyl group, R₂ represents a (C₁-C₆)alkyl group optionally substituted with the —NRₐR_{b} group, R₃ represents a hydrogen atom or an —NR_{c}R_{d} group, positioned in position 5 or 6 on the pyridine nucleus and m is an integer between 1 and 6.

9. The compound according to claim 8, in which R₂ represents a (C₁-C₆)alkyl group.

10. The compound according to claim 1, in which the double bond on the pyridine nucleus is in E or Z form.

11. The compound according to claim 1, in the form of a base or of an addition salt with an acid or in the form of a hydrate or of a solvate.

12. A compound chosen from one of the following: 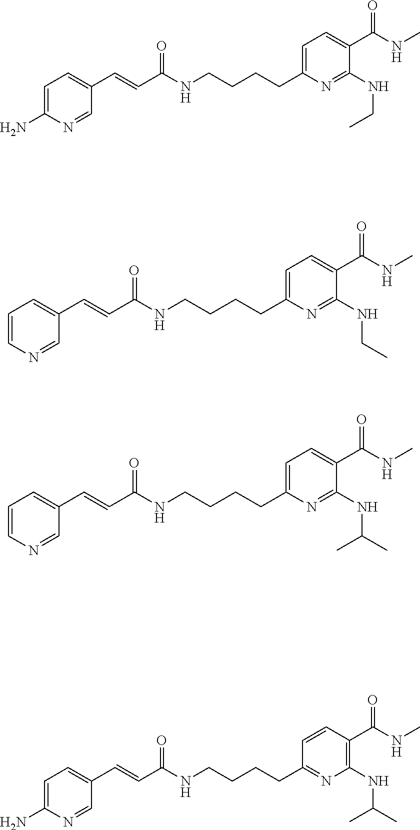
in which the double bond is in E form and which can be in the form of a base or of an addition salt with an acid or in the form of a hydrate or of a solvate.

13. A pharmaceutical composition comprising the compound of claim 1 and pharmaceutically acceptable salts thereof.

14. The pharmaceutical composition of claim 13, further comprising at least one pharmaceutically acceptable excipient.

15. An anticancer agent comprising the compound of claim 1.

16. A method of treating or preventing cancer in a patient in need thereof comprising administering to said patient a therapeutically effective amount of the pharmaceutical composition of claim 13.
